(19) United States
(12) Patent Application Publication
(10) Pub. No.: US 2023/0057356 A1
(43) Pub. Date: Feb. 23, 2023

(54) ENHANCEMENT OF UMBILICAL CORD MESENCHYMAL STEM CELL THERAPEUTIC ACTIVITY BY STIMULATORS OF T REGULATORY CELLS AND/OR CELLS EXPRESSING CD73

(71) Applicant: THERAPEUTIC SOLUTIONS INTERNATIONAL, INC., OCEANSIDE, CA (US)

(72) Inventors: Thomas C. ICHIM, Oceanside, CA (US); Famela RAMOS, OCEANSIDE, CA (US); James VELTMEYER, Oceanside, CA (US); Timothy G. DIXON, Oceanside, CA (US); Wais KAIHANI, Oceanside, CA (US)

(73) Assignee: THERAPEUTIC SOLUTIONS INTERNATIONAL, INC., OCEANSIDE, CA (US)

(21) Appl. No.: 17/820,789

(22) Filed: Aug. 18, 2022

Related U.S. Application Data
(60) Provisional application No. 63/234,627, filed on Aug. 18, 2021.

(51) Int. Cl. A61K 35/51; A61K 35/28; C12N 5/073; A61K 38/20

(52) U.S. Cl. CPC A61K 35/51; A61K 35/28; C12N 5/0605; A61K 38/2013; A61K 38/2086

(57) ABSTRACT

Disclosed are means, compositions of matter and protocols useful for treatment of COVID-19 and/or other inflammatory pathologies through stimulation of T regulatory cells and/or T cells expressing CD73 using administration of umbilical cord derived mesenchymal stem cells such as JadiCells. In one embodiment dosage of JadiCells needed to treat a patient is determined by the increase of T regulatory cells and/or CD73 expressing cells that are increased in number and/or activity subsequent to a test dose of JadiCells. In another embodiment stimulators of T regulatory cells and/or CD73 expressing T cells are utilized together with JadiCells in order to augment therapeutic activity. In some embodiments administration of JadiCell is performed with low dose interleukin-2 as a treatment for COVID-19 or other inflammatory related pathologies.

### CROSS-REFERENCE TO RELATED APPLICATIONS

This application is claims priority to U.S. Provisional Application Ser. No. 63/234,627, filed Aug. 18, 2021, which is incorporated by reference herein in its entirety.

### FIELD OF THE INVENTION

The invention pertains to the area of immunotherapy, more particularly the invention pertains to the area of inflammation specific immunotherapy, more specifically the invention pertains to treatment of COVID-19 using unexpected synergies.

### BACKGROUND OF THE INVENTION

Coronaviruses (CoVs) are a single strand sense of RNA which include four genera (alpha, beta, delta, and gamma) [1]. Infectivity of CoVs is mediated by the envelope spike (S) glycoprotein which binds to its cellular receptors angiotensin-converting enzyme 2 (ACE2) and dipeptidyl peptidase 4 (DPP4) for SARS-CoV and MERS-CoV, respectively [2, 3]. In the case of the novel COVID-19 virus, it is over 99% similar to SAR-CoV-2 which is a new type of beta genera. This is based on 10 sequenced samples collected from the original location of the outbreak [4]. SAR-2-CoV preferentially infects the type 2 pulmonary epithelial cells, in the lungs, which express ACE2 [5].

One of the main mechanisms of COVID-19 induced lethality is pulmonary failure.

Previous treatment approaches to COVID-19 have been numerous but with little having long term or consistent effects. For example, in one study, Sixteen SARS-CoV-2 infected patients requiring invasive mechanical ventilation for ARDS were treated with ruxolitinib in addition to standard treatment. Ruxolitinib treatment was well tolerated and 13 patients survived at least the first 28 days on treatment, which was the primary endpoint of the trial. Immediate start of ruxolitinib after deterioration was associated with improved outcome, as was a lymphocyte-to-neutrophils ratio above 0.07. Together, treatment with the janus-kinase inhibitor ruxolitinib is feasible and might be efficacious in COVID-19 induced ARDS patients requiring invasive mechanical ventilation [6, 7]. Unfortunately, ruxolitinib is known to be immune suppressive and actually dampens vaccine efficacy [8].

Treatment of COVID-19 with stem cells have previously been reported [9-13], however these treatments do not work in all patients. There is a need for elucidation of mechanisms of action and for novel synergies.

### SUMMARY

Preferred embodiments are directed to methods of stimulating treating COVID-19 and/or an inflammatory condition in an adult comprising administration of a population of umbilical cord derived mesenchymal stem cells together with a stimulator of T regulatory cells and/or CD73 expressing T cells.

Preferred embodiments include methods wherein said T regulatory cells express FOXP3.

Preferred embodiments include methods wherein said ongoing inflammation is associated with increased levels of TNF-alpha as compared to an age-matched control.

Preferred embodiments include methods wherein said ongoing inflammation is associated with increased levels of IL-1 as compared to an age-matched control.

Preferred embodiments include methods wherein said ongoing inflammation is associated with increased levels of IL-6 as compared to an age-matched control.

Preferred embodiments include methods wherein said inflammation is associated with stimulation of a toll like receptor (TLR).

Preferred embodiments include methods wherein said TLR is TLR-1.

Preferred embodiments include methods wherein said TLR is TLR-3.

Preferred embodiments include methods wherein said TLR is TLR-2.

Preferred embodiments include methods wherein said TLR is TLR-4.

Preferred embodiments include methods wherein said TLR is TLR-5.

Preferred embodiments include methods wherein said TLR is TLR-7/8.

Preferred embodiments include methods wherein said TLR is TLR-9.

Preferred embodiments include methods wherein said inflammation is associated with pulmonary failure.

Preferred embodiments include methods wherein said inflammation is associated with increased CRP compared to an age-matched control.

Preferred embodiments include methods wherein said umbilical cord mesenchymal stem cell is an isolated cell prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.

Preferred embodiments include methods wherein the isolated cell expresses CD29, CD73, CD90, CD166, SSEA4, CD9, CD44, CD146, and CD105.

Preferred embodiments include methods wherein the isolated cell does not express CD45, CD34, CD14, CD79, CD106, CD86, CD80, CD19, CD117, Stro-1, and HLA-DR.

Preferred embodiments include methods wherein the isolated cell is positive for SOX2.

Preferred embodiments include methods wherein the isolated cell is positive for OCT4.

Preferred embodiments include methods wherein the isolated cell is positive for SOX2 and OCT4.

Preferred embodiments include methods wherein the wherein the isolated cell is capable of differentiation into a cell type selected from the group consisting of adipocytes, chondrocytes, osteocytes, cardiomyocytes, endothelial cells, and myocytes.

Preferred embodiments include methods wherein the isolated cell produces exosomes expressing CD63, CD9, or CD63 and CD9.

Preferred embodiments include methods wherein culturing comprises culturing in a culture media that is free of animal components.

Preferred embodiments include cultures of differentiated cells derived from an isolated cell, wherein the culture of differentiated cells includes a cell type selected from the group consisting of adipocytes, chondrocytes, osteocytes, cardiomyocytes, endothelial cells, myocytes and combinations thereof.

Preferred isolated cells have been differentiated into an adipocyte cell.

Preferred isolated cells have been differentiated into a chondrocyte cell.

Preferred isolated cells have been differentiated into an osteocyte cell.

Preferred isolated cells have been differentiated into a cardiomyocyte cell.

Preferred isolated cells have been expanded into a cell culture.

Preferred embodiments include methods wherein said stimulator of T regulatory cells and/or CD73 expressing T cells increases the numbers of said T regulatory cells and/or CD73 expressing T cells.

Preferred embodiments include methods wherein said stimulator of T regulatory cells and/or CD73 expressing T cells increases the activity of said T regulatory cells and/or CD73 expressing T cells.

Preferred embodiments include methods wherein said activity of said T regulatory cells and/or CD73 expressing T cells is quantified by expression of TGF-beta.

Preferred embodiments include methods wherein said activity of said T regulatory cells and/or CD73 expressing T cells is quantified by expression of interleukin 1 receptor antagonist.

Preferred embodiments include methods wherein said activity of said T regulatory cells and/or CD73 expressing T cells is quantified by expression of leukemia inhibitor factor.

Preferred embodiments include methods wherein said activity of said T regulatory cells and/or CD73 expressing T cells is quantified by expression of placenta induced growth factor.

Preferred embodiments include methods wherein said activity of said T regulatory cells and/or CD73 expressing T cells is quantified by expression of angiopoietin.

Preferred embodiments include methods wherein said activity of said T regulatory cells and/or CD73 expressing T cells is quantified by expression of platelet derived growth factor.

Preferred embodiments include methods wherein said stimulator of Treg and/or CD73 expressing T cells is low dose interleukin-2.

Preferred embodiments include methods wherein said stimulator of Treg and/or CD73 expressing T cells is low dose interleukin-13.

Preferred embodiments include methods wherein said stimulator of Treg and/or CD73 expressing T cells is low dose interleukin-7.

Preferred embodiments include methods wherein said stimulator of Treg and/or CD73 expressing T cells is low dose interleukin-15.

Preferred embodiments include methods wherein said stimulator of Treg and/or CD73 expressing T cells is anti-CD3 antibodies.

### BRIEF DESCRIPTION OF THE DRAWINGS

FIG. 1 is a bar graph showing regulatory T cell enhancement (as measured by FoxP3 staining) in BALB/c mice based on administration of BM-MSC, Adipose MSC, or JadiCells.

FIG. 2 is a bar graph showing regulatory T cell enhancement (as measured by CD73 staining) in BALB/c mice based on administration of BM-MSC, Adipose MSC, or JadiCells.

FIG. 3 is a bar graph showing neutrophil numbers in BALB/c mice based on administration of either a) Antibodies, b) JadiCells, or c) JadiCells and antibody, being coadministered with poly IC after the mice were depleted of CD25+T reg cells.

FIG. 4 is a bar graph showing neutrophil numbers in BALB/c mice based on administration of either a) IL-2, b) JadiCells, or c) JadiCells and IL-2, being coadministered with poly IC after the mice were pretreated with IL-2.

### DETAILED DESCRIPTION OF THE INVENTION

The invention teaches that mesenchymal stem cells and particularly umbilical cord derived MSC induce an augmentation of Treg cells and/or CD73 expressing cells, which is associated with protection from inflammation. Furthermore the invention teaches that agents which increase Treg and/or CD73 T cell activity have a synergistic protective effect against inflammation when administered with stem cells.

For the purpose of the invention, one type of stem cell that is administered with agents that stimulate T regulatory cell and/or CD73 expressing cell activity such as IL-2. This type of stem cell, in one embodiment, is an isolated cell obtained from a subepithelial layer of a mammalian umbilical cord tissue capable of self-renewal and culture expansion is provided. Such a cell is capable of differentiation into a cell type such as, in one aspect for example, adipocytes, chondrocytes, osteocytes, cardiomyocytes, and the like. In another aspect, non-limiting examples of such cell types can include white, brown, or beige adipocytes, chondrocytes, osteocytes, cardiomyocytes, endothelial cells, myocytes, and the like, including combinations thereof. Other examples of such cell types can include neural progenitor cells, hepatocytes, islet cells, renal progenitor cells, and the like. A cross section of a human umbilical cord shows the umbilical artery (UA), the umbilical veins (UV), the Wharton's Jelly (WJ), and the subepithelial layer (SL). Isolated cells from the SL can have a variety of characteristic markers that distinguish them from cell previously isolated from umbilical cord samples. It should be noted that these isolated cells are not derived from the Wharton's Jelly, but rather from the SL.

Various cellular markers that are either present or absent can be utilized in the identification of these SL-derived cells, and as such, can be used to show the novelty of the isolated cells. For example, in one aspect, the isolated cell expresses at least three cell markers selected from CD29, CD73, CD90, CD146, CD166, SSEA4, CD9, CD44, CD146, or CD105, and the isolated cell does not express at least three cell markers selected from CD45, CD34, CD14, CD79, CD106, CD86, CD80, CD19, CD117, Stro-1, or HLA-DR. In another aspect, the isolated cell expresses at least five cell markers selected from CD29, CD73, CD90, CD146, CD166, SSEA4, CD9, CD44, CD146, or CD105. In another aspect, the isolated cell expresses at least eight cell markers selected from CD29, CD73, CD90, CD146, CD166, SSEA4, CD9, CD44, CD146, or CD105. In a yet another aspect, the isolated cell expresses at least CD29, CD73, CD90, CD166, SSEA4, CD9, CD44, CD146, and CD105. In another aspect, the isolated cell does not express at least five cell markers selected from CD45, CD34, CD14, CD79, CD106, CD86, CD80, CD19, CD117, Stro-1, or HLA-DR. In another aspect, the isolated cell does not express at least eight cell markers selected from CD45, CD34, CD14, CD79, CD106, CD86, CD80, CD19, CD117, Stro-1, or HLA-DR. In yet another aspect, the isolated cell does not express at least CD45, CD34, CD14, CD79, CD106, CD86, CD80, CD19, CD117, Stro-1, and HLA-DR. Additionally, in some aspects, the isolated cell can be positive for SOX2, OCT4, or both SOX2 and OCT4. In a further aspect, the isolated cell can produce exosomes expressing CD63, CD9, or both CD63 and CD9. A variety of techniques can be utilized to extract the isolated cells of the present disclosure from the SL, and any such technique that allows such extraction without significant damage to the cells is considered to be within the present scope. In one aspect, for example, a method of culturing stem cells from the SL of a mammalian umbilical cord can include dissecting the subepithelial layer from the umbilical cord. In one aspect, for example, umbilical cord tissue can be collected and washed to remove blood, Wharton's Jelly, and any other material associated with the SL. For example, in one non-limiting aspect the cord tissue can be washed multiple times in a solution of Phosphate-Buffered Saline (PBS) such as Dulbecco's Phosphate-Buffered Saline (DPBS). In some aspects the PBS can include a platelet lysate (i.e. 10% PRP lysate of platelet lysate). Any remaining Wharton's Jelly or gelatinous portion of the umbilical cord can then be removed and discarded. The remaining umbilical cord tissue (the SL) can then be placed interior side down on a substrate such that an interior side of the SL is in contact with the substrate. An entire dissected umbilical cord with the Wharton's Jelly removed can be placed directly onto the substrate, or the dissected umbilical cord can be cut into smaller sections (e.g. 1-3 mm) and these sections can be placed directly onto the substrate.

A variety of substrates are contemplated upon which the SL can be placed. In one aspect, for example, the substrate can be a solid polymeric material. One example of a solid polymeric material can include a cell culture dish. The cell culture dish can be made of a cell culture treated plastic as is known in the art. In one specific aspect, the SL can be placed upon the substrate of the cell culture dish without any additional pretreatment to the cell culture treated plastic. In another aspect, the substrate can be a semi-solid cell culture substrate. Such a substrate can include, for example, a semi-solid culture medium including an agar or other gelatinous base material.

The culture can then be cultured under either normoxic or hypoxic culture conditions for a period of time sufficient to establish primary cell cultures. (e.g. 3-7 days in some cases). After primary cell cultures have been established, the SL tissue is removed and discarded. Cells or stem cells are further cultured and expanded in larger culture flasks in either a normoxic or hypoxic culture conditions. While a variety of suitable cell culture media are contemplated, in one non-limiting example the media can be Dulbecco's Modified Eagle Medium (DMEM) glucose (500-6000 mg/mL) without phenol red, 1.times. glutamine, 1.times. NEAA, and 0.1-20% PRP lysate or platelet lysate. Another example of suitable media can include a base medium of DMEM low glucose without phenol red, 1.times. glutamine, 1.times. NEAA, 1000 units of heparin and 20% PRP lysate or platelet lysate. In another example, cells can be cultured directly onto a semi-solid substrate of DMEM low glucose without phenol red, 1.times. glutamine, 1.times. NEAA, and 20% PRP lysate or platelet lysate. In a further example, culture media can include a low glucose medium (500-1000 mg/mL) containing 1.times. Glutamine, 1.times. NEAA, 1000 units of heparin. In some aspects, the glucose can be 1000-4000 mg/mL, and in other aspects the glucose can be high glucose at 4000-6000 mg/mL. These media can also include 0.1%-20% PRP lysate or platelet lysate. In yet a further example, the culture medium can be a semi-solid with the substitution of acid-citrate-dextrose ACD in place of heparin, and containing low glucose medium (500-1000 mg/mL), intermediate glucose medium (1000-4000 mg/mL) or high glucose medium (4000-6000 mg/mL), and further containing 1.times. Glutamine, 1.times. NEAA, and 0.1%-20% PRP lysate or platelet lysate. In some aspects, the cells can be derived, subcultured, and/or passaged using TrypLE. In another aspect, the cells can be derived, subcultured, and/or passaged without the use of TrypLE or any other enzyme.

In other embodiments, JadiCells are administered concurrently with adjuvants that may stimulation therapeutic activity. Said Adjuvants include hCG, SSRISs and oxytocin.

The invention teaches the use of T regulatory cells to prevent, inhibit or reverse inflammation together with MSC such as JadiCells. In one embodiment, the invention provides for administration of exogenous T regulatory cells in a patient at risk inflammation or suffering from inflammation such as COVID-19. In another embodiment, the invention provides the use of agents which augment activity and/or number of endogenous T regulatory cells.

In some embodiments of the invention, stimulation of T regulatory cells in vivo is accomplished by administration of Aldesleukin (Proleukin, Novartis), which is a commercially available IL-2 licensed for the treatment of metastatic renal cell carcinoma in the UK. It is produced by recombinant DNA technology using an Escherichia coli strain, which contains a genetically engineered modification of the human IL-2 gene, and is administered either intravenously or subcutaneously (SC). Following short intervenous infusion, its pharmacokinetic profile is typified by high plasma concentrations, rapid distribution into the extravascular space and a rapid renal clearance. The recommended doses for continuous infusion and subcutaneous injection (as detailed in the Summary of Product Characteristics) are repeated cycles of 18×10⁶IU per m² per 24 hours for 5 days and repeated doses of 18×10⁶ IU, respectively. Peak plasma levels are reached in 2-6 hours after SC administration, with bioavailability of aldesleukin ranging between 31% and 47%. The process of absorption and elimination of subcutaneous aldesleukin is described by a one-compartment model, with a 45 min absorption half-life and an elimination half-life of 3-5 hours [14]. Natural IL-2 was first identified in 1976 as a growth factor for T lymphocytes. It is produced by human cluster designation (CD) 4+ and some CD8+T-cells and is synthesized mainly by activated T-cells, in particular CD4.sup.+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilit:ites the proliferation and differentiation of B-cells and the synthesis of immunoglobulin by B-cells, and stimulates the generation, proliferation and activation of natural killer (NK). IL-2 is known to play a central role in the generation of immune responses. In cancer clinical trials, high-dose recombinant IL-2 (e.g., IV bolus dose of 600,000 international units (IU)/kg every 8 hours for up to 14 doses) demonstrated antitumor activity in metastatic renal cell carcinoma (RCC) and metastatic melanoma. Accordingly, such high-dose IL-2 was approved for the treatment of metastatic RCC in Europe in 1989 and in the US in 1992. In 1998, approval was obtained to treat patients with metastatic melanoma. Recombinant human IL-2 (Aldesleukin) (Proleukin.RTM.-Novartis Inc. & Prometheus Labs Inc.) is currently approved by the United States Food and Drug Administration (US FDA). However, IL-2 has a dual function in the immune response in that it not only mediates expansion and activity of effector cells, but also is crucially involved in maintaining peripheral immune tolerance. A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD 3 complex, the expression of Fas ligand (FasL) and/or tumor necrosis factor (TNF) is induced, making the cells susceptible for Fas-mediated apoptosis. This process is IL-2 dependent and mediated via STATS. By the process of AICD in T lymphocytes tolerance can not only be established to self-antigens, but also to persistent antigens that are clearly not part of the host's makeup, such as tumor antigens.

In some embodiments of the invention, administration of angiogenic genes is performed to enhance efficacy of Treg cell therapy. Genes with angiogenic ability include: activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shingoingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, αf131 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF.

In one embodiment of the invention, patients suffering from COVID-19 are pretreated with 0.3×10⁶ IU of aldesleukin daily. Concentrations for clinical uses of aldesleukin could be used from the literature as described for other indications including heart failure [14], Wiskott-Aldrich syndrome [15], Graft Versus Host Disease [16, 17], lupus [18], type 1 diabetes [19-21] and are incorporated by reference. In some embodiments of the invention, administration of low doses of IL-2 in the form of aldesleukin every day at concentrations of 0.3×10⁶ to 3.0×10⁶ IU IL-2 per square meter of body surface area for 8 weeks, or in other embodiments repetitive 5-day courses of 1.0×10⁶ to 3.0×10⁶ IU IL-2. Various types of IL-2 may be utilized. Examples of IL-2 variants, recombinant IL-2, methods of IL-2 production, methods of IL-2 purification, methods of formulation, and the like are well known in the art and can be found, for example, at least in U.S. Pat. Nos. 4,530,787, 4,569,790, 4,572,798, 4,604,377, 4,748,234, 4,853,332, 4,959,314, 5,464,939, 5,229,109, 7,514,073, and 7,569,215, each of which is herein incorporated by reference in their entirety for all purposes. In some embodiments, low dose interleukin-2 is provided together with activators of coinhibitory molecules, otherwise known as checkpoints. Such coinhibitory molecules include CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, A2aR, and combinations thereof. In some embodiments of the invention, mesenchymal stem cells are co-administered. Protocols for use of MSC have been previously published and incorporated by reference [22, 23]. For example, mesenchymal stem cells of adipose [24-27], bone marrow [28-47], placental [48], amniotic membrane [49, 50], umbilical cord [51-57], menstrual blood [58], and lung [59, 60], origin, as well as conditioned media [61-68]. Additionally, the generation of Treg by mesenchymal stem cells is also described in the art, for which we are providing the following references to assist in the practice of the invention [69-97].

To provide prophylactic and/or therapeutic interventions, in the area of COVID-19 the invention teaches that it is important to delete/inactivate the T cell clone that are associated with stimulation of inflammation, as well as to block innate immune elements. This would be akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “mirror image” immune system was demonstrated to co-exist with the conventional immune system. Conventional T cells are activated by self-antigens to die in the thymus and conventional T cells that are not activated receive a survival signal [98]; the “mirror image”, T regulatory (Treg) cells are actually selected to live by encounter with self-antigens, and Treg cells that do not bind self antigens are deleted [99, 100]. In one embodiment of the invention, immature dendritic cells are administered in order to induce a state of immune modulation, including T regulatory cell generation by the immature dendritic cells. Utilization of immature dendritic cells to stimulate T regulatory cell proliferation and/or activity has been previously demonstrated and is incorporated by reference [101-107].

Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “anergized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

The invention teaches that utilization of tolerogenesis may be applied to Parkinson's Disease. Specific examples of tolerogenesis that occurs in adults includes settings such as pregnancy, cancer, and oral tolerance. In the situation of pregnancy, studies have demonstrated selective inactivation of maternal T cell clones that recognize fetal antigens occurs through a variety of mechanisms, including FasL expression on fetal and placental cells [108], antigen presentation in the context of PD1-L [109], and HLA-G interacting with immune inhibitory receptors such as ILT4 [110]. Accordingly, in some embodiments of the invention, the utilization of tolerogenic regimens is provided which mimic pregnancy associated tolerance. In one embodiment, such embodiments include fusion of tolerance promoting molecules with Parkinson's Disease associated antigens such as synuclein peptides. In other embodiments synuclein antigens are co-administered with tolerogenic molecules such as ILT-4, or IL-10, or HLA-G.

In pregnancy, “tolerogenic antigen presentation” occurs only through the indirect pathway of antigen presentation [111]. Other pathways of selective tolerogenesis in pregnancy include the stimulation of Treg cells, which have been demonstrated essential for successful pregnancy [112]. In the context of cancer, depletion of tumor specific T cells, while sparing of T cells with specificities to other antigens has been demonstrated by the tumor itself or tumor associated cells [113-116]. Additionally, Treg cells have been demonstrated to actively suppress anti-tumor T cells, perhaps as a “back up” mechanism of tumor immune evasion [117-119]. At a clinical level the ability of tumors to inhibit peripheral T cell activity has been associated in numerous studies with poor prognosis [120-122]. Oral tolerance is the process by which ingested antigens induce generation of antigen-specific TGF-beta producing cells (called “Th3” by some) [123-125], as well as Treg cells [126, 127]. Ingestion of antigen, including the autoantigen collagen II [128], has been shown to induce inhibition of both T and B cell responses in a specific manner [129, 130]. It appears that induction of regulatory cells, as well as deletion/anergy of effector cells is associated with antigen presentation in a tolerogenic manner [131]. Remission of disease in animal models of RA [132], multiple sclerosis [133], and type I diabetes [134], has been reported by oral administration of autoantigens. Furthermore, clinical trials have shown signals of efficacy of oral tolerance in autoimmune diseases such as rheumatoid arthritis [135], autoimmune uveitis [136], and multiple sclerosis [137]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction.

In some embodiments of the invention the generation of immature dendritic cells is performed by either coculture in vitro, or administration in vivo of T regulatory cells [138].

### Example 1: JadiCells Increase the Number of T Regulatory Cells

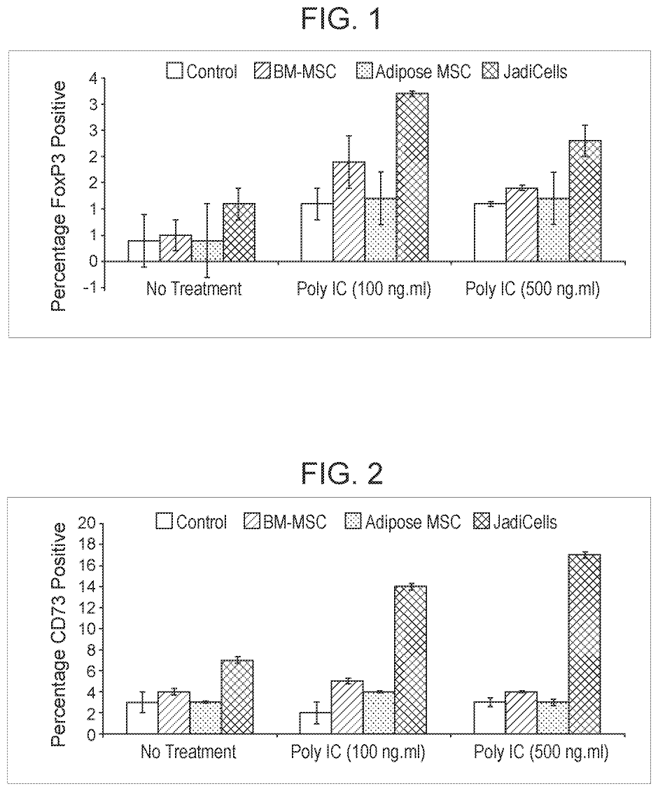

BALB/c mice were administered with 1 million of either bone marrow or adipose MSC or with JadiCells Intravenously at the same time as intraperitoneal injection of poly IC at the indicated concentrations. After a period of 3 days animals were sacrificed and T regulatory cells were measured by FoxP3 staining (Promega kit, following manufacturers instructions). The results are shown in FIG. 1.

### Example 2: JadiCells Increase the Number of CD73 T Cells

BALB/c mice were administered with 1 million of either bone marrow or adipose MSC or with JadiCells Intravenously at the same time as intraperitoneal injection of poly IC at the indicated concentrations. After a period of 3 days animals were sacrificed and T regulatory cells were measured by CD73 staining (Promega kit, following manufacturers instructions). The results are shown in FIG. 2.

### Example 3: Depletion of Treg Cells Decreases Therapeutic Potential of JadiCellsBALB/c

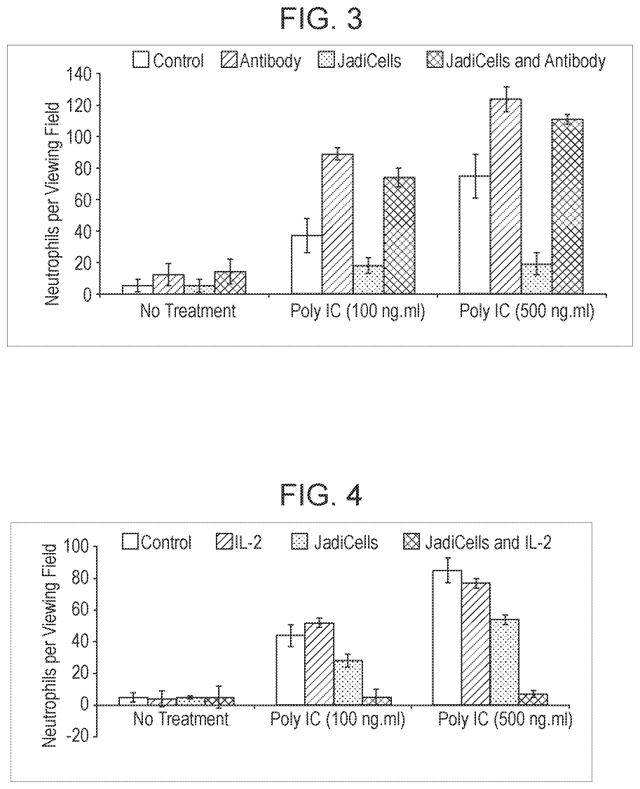

mice were depleted of CD25 T regulatory cells by administration once every two days of anti-CD25 antibody one week before initiation of experiment. Subsequently animals were administered with 1 million of either bone marrow or adipose MSC or with JadiCells Intravenously at the same time as intraperitoneal injection of poly IC at the indicated concentrations. After a period of 3 days animals were sacrificed and lung inflammation was assessed by neutrophils per viewing field. The results are shown in FIG. 3.

### Example 4: Low Dose IL-2 Increases Therapeutic Potential of JadiCells

BALB/c mice were treated with IL-2 (10 IU/mouse) once every two days one week before initiation of experiment. Subsequently animals were administered with 1 million of either bone marrow or adipose MSC or with JadiCells Intravenously at the same time as intraperitoneal injection of poly IC at the indicated concentrations. After a period of 3 days animals were sacrificed and lung inflammation was assessed by neutrophils per viewing field. The results are shown in FIG. 4.

### REFERENCES

ADDIN EN.REFLIST 1. de Wilde, A. H., et al., Host Factors in Coronavirus Replication. Curr Top Microbiol Immunol, 2018. 419: p. 1-42.
2. Raj, V. S., et al., Dipeptidyl peptidase 4 is a functional receptor for the emerging human coronavirus-EMC. Nature, 2013. 495(7440): p. 251-4.
3. Xia, S., et al., Inhibition of SARS-CoV-2 (previously 2019-nCoV) infection by a highly potent pan-coronavirus fusion inhibitor targeting its spike protein that harbors a high capacity to mediate membrane fusion. Cell Res, 2020.
4. Sevajol, M., et al., Insights into RNA synthesis, capping, and proofreading mechanisms of SARS-coronavirus. Virus Res, 2014. 194: p. 90-9.
5. Zou, X., et al., Single-cell RNA-seq data analysis on the receptor ACE2 expression reveals the potential risk of different human organs vulnerable to 2019-nCoV infection. Front Med, 2020.
6. Neubauer, A., et al., The janus-kinase inhibitor ruxolitinib in SARS-CoV-2 induced acute respiratory distress syndrome (ARDS). Leukemia, 2021.
7. Sarmiento, M., et al., Ruxolitinib for Severe COVID-19-Related Hyperinflammation in Nonresponders to Steroids. Acta Haematol, 2021: p. 1-7.
8. Guglielmelli, P., et al., Impaired response to first SARS-CoV-2 dose vaccination in myeloproliferative neoplasm patients receiving ruxolitinib. Am J Hematol, 2021.
9. Kesari, S., et al., Mesenchymal stem cells in the treatment of severe COVID-19. Transl Med Commun, 2021. 6(1): p. 16.
10. Wang, L., et al., Regulation of Inflammatory Cytokine Storms by Mesenchymal Stem Cells. Front Immunol, 2021. 12: p. 726909.
11. Yousefi Dehbidi, M., et al., Mesenchymal stem cells and their derived exosomes to combat Covid-19. Rev Med Virol, 2021: p. e2281.
12. Fernandez-Francos, S., et al., Mesenchymal Stem Cell-Based Therapy as an Alternative to the Treatment of Acute Respiratory Distress Syndrome: Current Evidence and Future Perspectives. Int J Mol Sci, 2021. 22(15).
13. da Silva, K. N., et al., Is there a place for mesenchymal stromal cell-based therapies in the therapeutic armamentarium against COVID-19? Stem Cell Res Ther, 2021. 12(1): p. 425.
14. Zhao, T. X., et al., Low-dose interleukin-2 in patients with stable ischaemic heart disease and acute coronary syndromes (LILACS): protocol and study rationale for a randomised, double-blind, placebo-controlled, phase I/II clinical trial. BMJ Open, 2018. 8(9): p. e022452.
15. Jyonouchi, S., et al., Phase I trial of low-dose interleukin 2 therapy in patients with Wiskott-Aldrich syndrome. Clin Immunol, 2017. 179: p. 47-53.
16. Asano, T., et al., Phase I/IIa Study of Low Dose Subcutaneous Interleukin-2 (IL-2) for Treatment of Refractory Chronic Graft Versus Host Disease. Acta Med Okayama, 2016. 70(5): p. 429-433.
17. Kennedy-Nasser, A. A., et al., Ultra low-dose IL-2 for GVHD prophylaxis after allogeneic hematopoietic stem cell transplantation mediates expansion of regulatory T cells without diminishing antiviral and antileukemic activity. Clin Cancer Res, 2014. 20(8): p. 2215-25.
18. Mizui, M. and G. C. Tsokos, Low-Dose IL-2 in the Treatment of Lupus. Curr Rheumatol Rep, 2016. 18(11): p. 68.
19. Todd, J. A., et al., Regulatory T Cell Responses in Participants with Type 1 Diabetes after a Single Dose of Interleukin-2: A Non-Randomised, Open Label, Adaptive Dose-Finding Trial. PLoS Med, 2016. 13(10): p. e1002139.
20. Pham, M.N., M.G. von Herrath, and J. L. Vela, Antigen-Specific Regulatory T Cells and Low Dose of IL-2 in Treatment of Type 1 Diabetes. Front Immunol, 2015. 6: p. 651.
21. Waldron-Lynch, F., et al., Rationale and study design of the Adaptive study of IL-2 dose on regulatory T cells in type 1 diabetes (DILT1D): a non-randomised, open label, adaptive dose finding trial. BMJ Open, 2014. 4(6): p. e005559.
22. von Bahr, V., et al., Mesenchymal stem cells may ameliorate inflammation in an ex vivo model of extracorporeal membrane oxygenation. Perfusion, 2019. 34(1_suppl): p. 15-21.
23. Huppert, L.A., K.D. Liu, and M.A. Matthay, Therapeutic potential of mesenchymal stromal cells in the treatment of ARDS. Transfusion, 2019. 59(S1): p. 869-875.
24. Jung, Y. J., et al., The effect of human adipose-derived stem cells on lipopolysaccharide- induced acute respiratory distress syndrome in mice. Ann Transl Med, 2019. 7(22): p. 674.
25. Chen, C. H., et al., Effective protection against acute respiratory distress syndrome/sepsis injury by combined adipose-derived mesenchymal stem cells and preactivated disaggregated platelets. Oncotarget, 2017. 8(47): p. 82415-82429.
26. Lu, H., et al., Pulmonary Retention of Adipose Stromal Cells Following Intravenous Delivery Is Markedly Altered in the Presence of ARDS. Cell Transplant, 2016. 25(9): p. 1635-1643.
27. Zheng, G., et al., Treatment of acute respiratory distress syndrome with allogeneic adipose-derived mesenchymal stem cells: a randomized, placebo-controlled pilot study. Respir Res, 2014. 15: p. 39.
28. Lu, Z., et al., Mesenchymal stem cells induce dendritic cell immune tolerance via paracrine hepatocyte growth factor to alleviate acute lung injury. Stem Cell Res Ther, 2019. 10(1): p. 372.
29. Silva, J. D., et al., Mesenchymal Stromal Cells Are More Effective Than Their Extracellular Vesicles at Reducing Lung Injury Regardless of Acute Respiratory Distress Syndrome Etiology. Stem Cells Int, 2019. 2019: p. 8262849.
30. Xu, A. L., et al., Mesenchymal Stem Cells Reconditioned in Their Own Serum Exhibit Augmented Therapeutic Properties in the Setting of Acute Respiratory Distress Syndrome. Stem Cells Transl Med, 2019. 8(10): p. 1092-1106.
31. Cardenes, N., et al., Cell therapy for ARDS: efficacy of endobronchial versus intravenous administration and biodistribution of MAPCs in a large animal model. BMJ Open Respir Res, 2019. 6(1): p. e000308.
32. Li, L., et al., Mesenchymal stem cells with downregulated Hippo signaling attenuate lung injury in mice with lipopolysaccharideinduced acute respiratory distress syndrome. Int J Mol Med, 2019. 43(3): p. 1241-1252.
33. Mokhber Dezfouli, M. R., et al., Intrapulmonary autologous transplant of bone marrow- derived mesenchymal stromal cells improves lipopolysaccharide-induced acute respiratory distress syndrome in rabbit. Crit Care, 2018. 22(1): p. 353.
34. Schwede, M., et al., Effects of bone marrow-derived mesenchymal stromal cells on gene expression in human alveolar type II cells exposed to TNF-alpha, IL-1 beta, and IFN- gamma. Physiol Rep, 2018. 6(16): p. e13831.
35. Masterson, C., et al., Syndecan-2-positive, Bone Marrow-derived Human Mesenchymal Stromal Cells Attenuate Bacterial-induced Acute Lung Injury and Enhance Resolution of Ventilator-induced Lung Injury in Rats. Anesthesiology, 2018. 129(3): p. 502-516.
36. Park, J., et al., Expression profile of microRNAs following bone marrow-derived mesenchymal stem cell treatment in lipopolysaccharide-induced acute lung injury. Exp Ther Med, 2018. 15(6): p. 5495-5502.
37. Pedrazza, L., et al., Mesenchymal stem cells improves survival in LPS-induced acute lung injury acting through inhibition of NETs formation. J Cell Physiol, 2017. 232(12): p. 3552-3564.
38. Yang, Y., et al., The Vascular Endothelial Growth Factors-Expressing Character of Mesenchymal Stem Cells Plays a Positive Role in Treatment of Acute Lung Injury In Vivo. Mediators Inflamm, 2016. 2016: p. 2347938.
39. Moodley, Y., et al., Human mesenchymal stem cells attenuate early damage in a ventilated pig model of acute lung injury. Stem Cell Res, 2016. 17(1): p. 25-31.
40. Hayes, M., et al., Mesenchymal stromal cells are more effective than the MSC secretome in diminishing injury and enhancing recovery following ventilator-induced lung injury. Intensive Care Med Exp, 2015. 3(1): p. 29.
41. Monsel, A., et al., Therapeutic Effects of Human Mesenchymal Stem Cell-derived Microvesicles in Severe Pneumonia in Mice. Am J Respir Crit Care Med, 2015. 192(3): p. 324-36.
42. Hao, Q., et al., Study of Bone Marrow and Embryonic Stem Cell-Derived Human Mesenchymal Stem Cells for Treatment of Escherichia coli Endotoxin-Induced Acute Lung Injury in Mice. Stem Cells Transl Med, 2015. 4(7): p. 832-40.
43. Devaney, J., et al., Human mesenchymal stromal cells decrease the severity of acute lung injury induced by E. coli in the rat. Thorax, 2015. 70(7): p. 625-35.
44. Asmussen, S., et al., Human mesenchymal stem cells reduce the severity of acute lung injury in a sheep model of bacterial pneumonia. Thorax, 2014. 69(9): p. 819-25.
45. Shalaby, S. M., et al., Mesenchymal stromal cell injection protects against oxidative stress in Escherichia coli-induced acute lung injury in mice. Cytotherapy, 2014. 16(6): p. 764-75.
46. Bustos, M. L., et al., Activation of human mesenchymal stem cells impacts their therapeutic abilities in lung injury by increasing interleukin (IL)-10 and IL-1RN levels. Stem Cells Transl Med, 2013. 2(11): p. 884-95.
47. Rojas, M., et al., Infusion of freshly isolated autologous bone marrow derived mononuclear cells prevents endotoxin-induced lung injury in an ex-vivo perfused swine model. Stem Cell Res Ther, 2013. 4(2): p. 26.
48. Yan, X., et al., Nrf2/Keap1/ARE Signaling Mediated an Antioxidative Protection of Human Placental Mesenchymal Stem Cells of Fetal Origin in Alveolar Epithelial Cells. Oxid Med Cell Longev, 2019. 2019: p. 2654910.
49. Cui, P., et al., Human amnion-derived mesenchymal stem cells alleviate lung injury induced by white smoke inhalation in rats. Stem Cell Res Ther, 2018. 9(1): p. 101.
50. Zhang, S., et al., Nrf2 transfection enhances the efficacy of human amniotic mesenchymal stem cells to repair lung injury induced by lipopolysaccharide. J Cell Biochem, 2018. 119(2): p. 1627-1636.
51. Huang, Z., et al., Transcriptomic analysis of lung tissues after hUC-MSCs and FTY720 treatment of lipopolysaccharide-induced acute lung injury in mouse models. Int Immunopharmacol, 2018. 63: p. 26-34.
52. Xuan, Y. Y., et al., Human Mesenchymal Stem/Stromal Cells From Human Umbilical Cord Ameliorate Acute Respiratory Distress Syndrome in Rats: Factors to Consider. Crit Care Med, 2017. 45(7): p. e736-e737.
53. Lee, F. Y., et al., Xenogeneic human umbilical cord-derived mesenchymal stem cells reduce mortality in rats with acute respiratory distress syndrome complicated by sepsis. Oncotarget, 2017. 8(28): p. 45626-45642.
54. Zhu, H., et al., Therapeutic Effects of Human Umbilical Cord-Derived Mesenchymal Stem Cells in Acute Lung Injury Mice. Sci Rep, 2017. 7: p. 39889.
55. Curley, G. F., et al., Cryopreserved, Xeno-Free Human Umbilical Cord Mesenchymal Stromal Cells Reduce Lung Injury Severity and Bacterial Burden in Rodent Escherichia coli-Induced Acute Respiratory Distress Syndrome. Crit Care Med, 2017. 45(2): p. e202-e212.
56. Chang, Y., et al., Intratracheal administration of umbilical cord blood-derived mesenchymal stem cells in a patient with acute respiratory distress syndrome. J Korean Med Sci, 2014. 29(3): p. 438-40.
57. Moodley, Y., et al., Human umbilical cord mesenchymal stem cells reduce fibrosis of bleomycin-induced lung injury. Am J Pathol, 2009. 175(1): p. 303-13.
58. Xiang, B., et al., Transplantation of Menstrual Blood-Derived Mesenchymal Stem Cells Promotes the Repair of LPS-Induced Acute Lung Injury. Int J Mol Sci, 2017. 18(4).
59. Wang, L., et al., Lung-Resident Mesenchymal Stem Cells Promote Repair of LPS-Induced Acute Lung Injury via Regulating the Balance of Regulatory T cells and Th17 cells. Inflammation, 2019. 42(1): p. 199-210.
60. Silva, J. D., et al., Mesenchymal Stem Cells From Bone Marrow, Adipose Tissue, and Lung Tissue Differentially Mitigate Lung and Distal Organ Damage in Experimental Acute Respiratory Distress Syndrome. Crit Care Med, 2018. 46(2): p. e132-e140.
61. Su, V.Y., et al., Mesenchymal Stem Cell-Conditioned Medium Induces Neutrophil Apoptosis Associated with Inhibition of the NF-kappaB Pathway in Endotoxin-Induced Acute Lung Injury. Int J Mol Sci, 2019. 20(9).
62. Mohammadipoor, A., et al., Therapeutic potential of products derived from mesenchymal stem/stromal cells in pulmonary disease. Respir Res, 2018. 19(1): p. 218.
63. Lee, J.H., J. Park, and J.W. Lee, Therapeutic use of mesenchymal stem cell-derived extracellular vesicles in acute lung injury. Transfusion, 2019. 59(S1): p. 876-883.
64. Abreu, S. C., D. J. Weiss, and P. R. Rocco, Extracellular vesicles derived from mesenchymal stromal cells: a therapeutic option in respiratory diseases? Stem Cell Res Ther, 2016. 7(1): p. 53.
65. Monsel, A., et al., Mesenchymal stem cell derived secretome and extracellular vesicles for acute lung injury and other inflammatory lung diseases. Expert Opin Biol Ther, 2016. 16(7): p. 859-71.
66. Liu, F. B., Q. Lin, and Z. W. Liu, A study on the role of apoptotic human umbilical cord mesenchymal stem cells in bleomycin-induced acute lung injury in rat models. Eur Rev Med Pharmacol Sci, 2016. 20(5): p. 969-82.
67. Chen, J., et al., Mesenchymal Stem Cell Conditioned Medium Promotes Proliferation and Migration of Alveolar Epithelial Cells under Septic Conditions In Vitro via the JNK-P38 Signaling Pathway. Cell Physiol Biochem, 2015. 37(5): p. 1830-46.
68. Ionescu, L., et al., Stem cell conditioned medium improves acute lung injury in mice: in vivo evidence for stem cell paracrine action. Am J Physiol Lung Cell Mol Physiol, 2012. 303(11): p. L967-77.
69. Court, A. C., et al., Mitochondrial transfer from MSCs to T cells induces Treg differentiation and restricts inflammatory response. EMBO Rep, 2020. 21(2): p. e48052.
70. Liu, Y., et al., Human umbilical cord mesenchymal stem cells confer potent immunosuppressive effects in Sjogren's syndrome by inducing regulatory T cells. Mod Rheumatol, 2020: p. 1-11.
71. Fakhimi, M., et al., Helios, CD73 and CD39 Induction in Regulatory T Cells Exposed to Adipose Derived Mesenchymal Stem Cells. Cell J, 2020. 22(2): p. 236-244.
72. Khosravi, M., et al., Induction of CD4(+)CD25(+)FOXP3(+) regulatory T cells by mesenchymal stem cells is associated with modulation of ubiquitination factors and TSDR demethylation. Stem Cell Res Ther, 2018. 9(1): p. 273.
73. Roux, C., et al., Immunosuppressive Mesenchymal Stromal Cells Derived from Human- Induced Pluripotent Stem Cells Induce Human Regulatory T Cells In Vitro and In Vivo. Front Immunol, 2017. 8: p. 1991.
74. Miyagawa, I., et al., Regulatory Mechanism of The Induction of Regulatory T Cells through Growth Factors Released by Human Mesenchymal Stem Cells. Crit Rev Immunol, 2018. 38(6): p. 471-478.
75. Khosravi, M., et al., Induction of CD4(+)CD25(+)Foxp3(+) regulatory T cells by mesenchymal stem cells is associated with RUNX complex factors. Immunol Res, 2018. 66(1): p. 207-218.
76. Khosravi, M., et al., Mesenchymal stem cells can induce regulatory T cells via modulating miR-126a but not miR-10a. Gene, 2017. 627: p. 327-336.
77. Lee, H. J., et al., ICOSL expression in human bone marrow-derived mesenchymal stem cells promotes induction of regulatory T cells. Sci Rep, 2017. 7: p. 44486.
78. Chen, C., et al., Mesenchymal stem cells upregulate Treg cells via sHLA-G in SLE patients. Int Immunopharmacol, 2017. 44: p. 234-241.
79. Lim, J. Y., et al., Enhanced immunoregulation of mesenchymal stem cells by IL-10- producing type 1 regulatory T cells in collagen-induced arthritis. Sci Rep, 2016. 6: p. 26851.
80. Lee, E. S., et al., Adoptive Transfer of Treg Cells Combined with Mesenchymal Stem Cells Facilitates Repopulation of Endogenous Treg Cells in a Murine Acute GVHD Model. PLoS One, 2015. 10(9): p. e0138846.
81. Cahill, E. F., et al., Jagged-1 is required for the expansion of CD4+CD25+FoxP3+ regulatory T cells and tolerogenic dendritic cells by murine mesenchymal stromal cells. Stem Cell Res Ther, 2015. 6: p. 19.
82. Wang, Z. X., et al., Mesenchymal stem cells alleviate atherosclerosis by elevating number and function of CD4(+)CD25 (+)FOXP3 (+) regulatory T-cells and inhibiting macrophage foam cell formation. Mol Cell Biochem, 2015. 400(1-2): p. 163-72.
83. Takahashi, T., et al., Multipotent mesenchymal stromal cells synergize with costimulation blockade in the inhibition of immune responses and the induction of Foxp3+ regulatory T cells. Stem Cells Transl Med, 2014. 3(12): p. 1484-94.
84. Frazier, T. P., et al., Human adipose-derived stromal/stem cells induce functional CD4+CD25+FoxP3+CD127- regulatory T cells under low oxygen culture conditions. Stem Cells Dev, 2014. 23(9): p. 968-77.
85. Li, J. G., et al., Human mesenchymal stem cells elevate CD4+CD25+CD127low/-regulatory T cells of asthmatic patients via heme oxygenase-1. Iran J Allergy Asthma Immunol, 2013. 12(3): p. 228-35.
86. Luz-Crawford, P., et al., Mesenchymal stem cells generate a CD4+CD25+Foxp3+ regulatory T cell population during the differentiation process of Thi and Th17 cells. Stem Cell Res Ther, 2013. 4(3): p. 65.
87. Melief, S. M., et al., Multipotent stromal cells induce human regulatory T cells through a novel pathway involving skewing of monocytes toward anti-inflammatory macrophages. Stem Cells, 2013. 31(9): p. 1980-91.
88. Erkers, T., et al., Decidual stromal cells promote regulatory T cells and suppress alloreactivity in a cell contact-dependent manner. Stem Cells Dev, 2013. 22(19): p. 2596-605.
89. Engela, A. U., et al., Human adipose-tissue derived mesenchymal stem cells induce functional de-novo regulatory T cells with methylated FOXP3 gene DNA. Clin Exp Immunol, 2013. 173(2): p. 343-54.
90. Tasso, R., et al., Mesenchymal stem cells induce functionally active T-regulatory lymphocytes in a paracrine fashion and ameliorate experimental autoimmune uveitis. Invest Ophthalmol Vis Sci, 2012. 53(2): p. 786-93.
91. Choi, Y. S., J. A. Jeong, and D. S. Lim, Mesenchymal stem cell-mediated immature dendritic cells induce regulatory T cell-based immunosuppressive effect. Immunol Invest, 2012. 41(2): p. 214-29.
92. Sun, J., et al., Intrapulmonary delivery of human umbilical cord mesenchymal stem cells attenuates acute lung injury by expanding CD4+CD25+Forkhead Boxp3 (FOXP3)+ regulatory T cells and balancing anti-and pro-inflammatory factors. Cell Physiol Biochem, 2011. 27(5): p. 587-96.
93. Madec, A. M., et al., Mesenchymal stem cells protect NOD mice from diabetes by inducing regulatory T cells. Diabetologia, 2009. 52(7): p. 1391-9.
94. English, K., et al., Cell contact, prostaglandin E(2) and transforming growth factor beta 1 play non-redundant roles in human mesenchymal stem cell induction of CD4+CD25(High) forkhead box P3+ regulatory T cells. Clin Exp Immunol, 2009. 156(1): p. 149-60.
95. Gonzalez-Rey, E., et al., Human adipose-derived mesenchymal stem cells reduce inflammatory and T cell responses and induce regulatory T cells in vitro in rheumatoid arthritis. Ann Rheum Dis, 2010. 69(1): p. 241-8.
96. Casiraghi, F., et al., Pretransplant infusion of mesenchymal stem cells prolongs the survival of a semiallogeneic heart transplant through the generation of regulatory T cells. J Immunol, 2008. 181(6): p. 3933-46.
97. Prevosto, C., et al., Generation of CD4+ or CD8+ regulatory T cells upon mesenchymal stem cell-lymphocyte interaction. Haematologica, 2007. 92(7): p. 881-8.
98. Ramsdell, F. and B. J. Fowlkes, Clonal deletion versus clonal anergy: the role of the thymus in inducing self tolerance. Science, 1990. 248(4961): p. 1342-8.
99. Apostolou, I., et al., Origin of regulatory T cells with known specificity for antigen. Nat Immunol, 2002. 3(8): p. 756-63.
100. Aschenbrenner, K., et al., Selection of Foxp3+ regulatory T cells specific for self antigen expressed and presented by Aire+medullary thymic epithelial cells. Nat Immunol, 2007. 8(4): p. 351-8.
101. Cong, Y., et al., Generation of antigen-specific, Foxp3-expressing CD4+ regulatory T cells by inhibition of APC proteosome function. J Immunol, 2005. 174(5): p. 2787-95.
102. Buckland, M., et al., Aspirin-treated human DCs up-regulate ILT-3 and induce hyporesponsiveness and regulatory activity in responder T cells. Am J Transplant, 2006. 6(9): p. 2046-59.
103. Jin, Y., et al., Induction of auto-reactive regulatory T cells by stimulation with immature autologous dendritic cells. Immunol Invest, 2007. 36(2): p. 213-32.
104. Gandhi, R., D. E. Anderson, and H. L. Weiner, Cutting Edge: Immature human dendritic cells express latency-associated peptide and inhibit T cell activation in a TGF-beta- dependent manner. J Immunol, 2007. 178(7): p. 4017-21.
105. Gaudreau, S., et al., Granulocyte-macrophage colony-stimulating factor prevents diabetes development in NOD mice by inducing tolerogenic dendritic cells that sustain the suppressive function of CD4+CD25+ regulatory T cells. J Immunol, 2007. 179(6): p. 3638-47.
106. Zhang, X., et al., Generation of therapeutic dendritic cells and regulatory T cells for preventing allogeneic cardiac graft rejection. Clin Immunol, 2008. 127(3): p. 313-21.
107. Marguti, I., et al., Expansion of CD4+CD25+Foxp3+T cells by bone marrow-derived dendritic cells. Immunology, 2009. 127(1): p. 50-61.
108. Vacchio, M. S. and R. J. Hodes, Fetal expression of Fas ligand is necessary and sufficient for induction of CD8 T cell tolerance to the fetal antigen H-Y during pregnancy. J Immunol, 2005. 174(8): p. 4657-61.
109. D′Addio, F., et al., The link between the PDL] costimulatory pathway and Th17 in fetomaternal tolerance. J Immunol, 2011. 187(9): p. 4530-41.
110. Kuroki, K. and K. Maenaka, Immune modulation of HLA-G dimer in maternal-fetal interface. Eur J Immunol, 2007. 37(7): p. 1727-9.
111. Erlebacher, A., et al., Constraints in antigen presentation severely restrict T cell recognition of the allogeneic fetus. J Clin Invest, 2007. 117(5): p. 1399-411.
112. Chen, T., et al., Self-specific memory regulatory T cells protect embryos at implantation in mice. J Immunol, 2013. 191(5): p. 2273-81.
113. Harimoto, H., et al., Inactivation of tumor-specific CD8(+) CTLs by tumor-infiltrating tolerogenic dendritic cells. Immunol Cell Biol, 2013. 91(9): p. 545-55.
114. Ney, J. T., et al., Autochthonous liver tumors induce systemic T cell tolerance associated with T cell receptor down-modulation. Hepatology, 2009. 49(2): p. 471-81.
115. Cheung, A. F., et al., Regulated expression of a tumor-associated antigen reveals multiple levels of T-cell tolerance in a mouse model of lung cancer. Cancer Res, 2008. 68(22): p. 9459-68.
116. Bai, A., et al., Rapid tolerization of virus-activated tumor-specific CD8+T cells in prostate tumors of TRAMP mice. Proc Natl Acad Sci USA, 2008. 105(35): p. 13003-8.
117. Jacobs, J. F., et al., Regulatory T cells in melanoma: the final hurdle towards effective immunotherapy? Lancet Oncol, 2012. 13(1): p. e32-42.
118. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.
119. Donkor, M. K., et al., T cell surveillance of oncogene-induced prostate cancer is impeded by T cell-derived TGF-betal cytokine. Immunity, 2011. 35(1): p. 123-34.
120. Whiteside, T. L., Down-regulation of zeta-chain expression in T cells: a biomarker of prognosis in cancer? Cancer Immunol Immunother, 2004. 53(10): p. 865-78.
121. Whiteside, T. L., Signaling defects in T lymphocytes of patients with malignancy. Cancer Immunol Immunother, 1999. 48(7): p. 346-52.
122. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.
123. Faria, A. M. and H. L. Weiner, Oral tolerance. Immunol Rev, 2005. 206: p. 232-59.
124. Weiner, H. L., Induction and mechanism of action of transforming growth factor-beta- secreting Th3 regulatory cells. Immunol Rev, 2001. 182: p. 207-14.
125. Fukaura, H., et al., Induction of circulating myelin basic protein and proteolipid protein- specific transforming growth factor-betal-secreting Th3 T cells by oral administration of myelin in multiple sclerosis patients. J Clin Invest, 1996. 98(1): p. 70-7.
126. Palomares, O., et al., Induction and maintenance of allergen-specific FOXP3+Treg cells in human tonsils as potential first-line organs of oral tolerance. J Allergy Clin Immunol, 2012. 129(2): p. 510-20, 520 el-9.
127. Yamashita, H., et al., Overcoming food allergy through acquired tolerance conferred by transfer of Tregs in a murine model. Allergy, 2012. 67(2): p. 201-9.
128. Park, K. S., et al., Type II collagen oral tolerance; mechanism and role in collagen- induced arthritis and rheumatoid arthritis. Mod Rheumatol, 2009.
129. Womer, K. L., et al., A pilot study on the immunological effects of oral administration of donor major histocompatibility complex class II peptides in renal transplant recipients. Clin Transplant, 2008. 22(6): p. 754-9.
130. Faria, A. M. and H. L. Weiner, Oral tolerance: therapeutic implications for autoimmune diseases. Clin Dev Immunol, 2006. 13(2-4): p. 143-57.
131. Park, M. J., et al., A distinct tolerogenic subset of splenic ID0(+)CD11b(+) dendritic cells from orally tolerized mice is responsible for induction of systemic immune tolerance and suppression of collagen-induced arthritis. Cell Immunol, 2012. 278(1-2): p. 45-54.
132. Thompson, H. S., et al., Suppression of collagen induced arthritis by oral administration of type II collagen: changes in immune and arthritic responses mediated by active peripheral suppression. Autoimmunity, 1993. 16(3): p. 189-99.
133. Song, F., et al., The thymus plays a role in oral tolerance induction in experimental autoimmune encephalomyelitis. Ann N Y Acad Sci, 2004. 1029: p. 402-4.
134. Hanninen, A. and L. C. Harrison, Mucosal tolerance to prevent type1 diabetes: can the outcome be improved in humans? Rev Diabet Stud, 2004. 1(3): p. 113-21.
135. Wei, W., et al., A multicenter, double-blind, randomized, controlled phase III clinical trial of chicken type II collagen in rheumatoid arthritis. Arthritis Res Ther, 2009. 11(6): p. R180.
136. Thurau, S. R., et al., Molecular mimicry as a therapeutic approach for an autoimmune disease: oral treatment of uveitis patients with an MHC-peptide crossreactive with autoantigen first results. Immunol Lett, 1997. 57(1-3): p. 193-201.
137. Weiner, H. L., et al., Double-blind pilot trial of oral tolerization with myelin antigens in multiple sclerosis. Science, 1993. 259(5099): p. 1321-4.
138. Onishi, Y., et al., Foxp3+natural regulatory T cells preferentially form aggregates on dendritic cells in vitro and actively inhibit their maturation. Proc Natl Acad Sci USA, 2008. 105(29): p. 10113-8.

1. A method of stimulating treating COVID-19 and/or an inflammatory condition in an adult comprising administration of a population of umbilical cord derived mesenchymal stem cells together with a stimulator of T regulatory cells and/or CD73 expressing T cells.

2. The method of claim 1, wherein said T regulatory cells express FOXP3.

3. The method of claim 2, wherein said ongoing inflammation is associated with increased levels of TNF-alpha as compared to an age-matched control.

4. The method of claim 2, wherein said ongoing inflammation is associated with increased levels of IL-1 as compared to an age-matched control.

5. The method of claim 2, wherein said ongoing inflammation is associated with increased levels of IL-6 as compared to an age-matched control.

6. The method of claim 2, wherein said inflammation is associated with stimulation of toll like receptor (TLR) 1-9.

7. The method of claim 1, wherein said umbilical cord mesenchymal stem cell is an isolated cell prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.

8. The isolated cell of claim 7, wherein the isolated cell produces exosomes expressing CD63, CD9, or CD63 and CD9.

9. The isolated cell of claim 7, wherein culturing comprises culturing in a culture media that is free of animal components.

10. The method of claim 1, wherein said stimulator of T regulatory cells and/or CD73 expressing T cells increases the numbers of said T regulatory cells and/or CD73 expressing T cells.

11. The method of claim 1, wherein said stimulator of T regulatory cells and/or CD73 expressing T cells increases the activity of said T regulatory cells and/or CD73 expressing T cells.

12. The method of claim 11, wherein said activity of said T regulatory cells and/or CD73 expressing T cells is quantified by expression of TGF-beta.

13. The method of claim 11, wherein said activity of said T regulatory cells and/or CD73 expressing T cells is quantified by expression of interleukin 1 receptor antagonist.

14. The method of claim 11, wherein said activity of said T regulatory cells and/or CD73 expressing T cells is quantified by expression of leukemia inhibitor factor.

15. The method of claim 11, wherein said activity of said T regulatory cells and/or CD73 expressing T cells is quantified by expression of placenta induced growth factor.

16. The method of claim 11, wherein said activity of said T regulatory cells and/or CD73 expressing T cells is quantified by expression of angiopoietin.

17. The method of claim 11, wherein said activity of said T regulatory cells and/or CD73 expressing T cells is quantified by expression of platelet derived growth factor.

18. The method of claim 1, wherein said stimulator of Treg and/or CD73 expressing T cells is low dose interleukin-2.

19. The method of claim 1, wherein said stimulator of Treg and/or CD73 expressing T cells is low dose interleukin-13.

20. The method of claim 1, wherein said stimulator of Treg and/or CD73 expressing T cells is low dose interleukin-15.
